(19) United States
(12) Patent Application Publication
(10) Pub. No.: US 2023/0310107 A1
(43) Pub. Date: Oct. 5, 2023

(54) MAGNETICALLY ATTACHABLE TISSUE CLIPS FOR USE IN ENDOSCOPIC SUBMUCOSAL DISSECTION

(71) Applicant: Covidien LP, Mansfield, MA (US)

(72) Inventors: William J. Camisa, Santa Clara, CA (US); Shawn C. Daniel, Mountlake Terrace, WA (US); Ryan W. Bertwell, San Jose, CA (US)

(21) Appl. No.: 18/010,537

(22) PCT Filed: Jul. 15, 2021
(86) PCT No.: PCT/US21/41750

§ 371 Date: Dec. 15, 2022

Related U.S. Application Data
(60) Provisional application No. 63/052,558, filed on Jul. 16, 2020.

(51) Int. Cl. A61B 34/00; A61B 17/02; A61B 17/122

(52) U.S. Cl. CPC A61B 34/73; A61B 17/0218; A61B 17/122; A61B 2017/00876

(57) ABSTRACT

A surgical clip for use in endoscopic submucosal dissection includes first and second jaws configured to move between an open configuration and a closed configuration to grasp tissue therebetween, a spherical magnet, and a tether coupling the spherical magnet to the first jaw and/or the second jaw.

### CROSS-REFERENCE TO RELATED APPLICATION

This application claims the benefit of and priority to U.S. Provisional Patent Application No. 63/052,558, filed Jul. 16, 2020, the entire contents of which are incorporated by reference herein.

### FIELD

The present technology is related generally to surgical tissue clips used in endoscopic submucosal dissection.

### BACKGROUND

Endoscopic dissection has been accepted as a first choice of the treatment for early stage GI carcinomas because of less invasiveness and lower cost. Endoscopic submucosal dissection (ESD) allows for an en bloc resection and accurate histopathological diagnosis regardless of the size, an existence of severe fibrosis at submucosal layer, and location of a lesion. One of the benefits of ESD is lower recurrence rates compared to endoscopic mucosal resection (EMR). During some ESDs, jaw members of a hemostatic clip are engaged to tissue. After performing the dissection, the jaw members are disengaged from the tissue.

### SUMMARY

In accordance with an aspect of the disclosure, a surgical clip for use in endoscopic submucosal dissection is provided and includes first and second jaws configured to move between an open configuration and a closed configuration to grasp tissue therebetween, a spherical magnet, and a tether coupling the spherical magnet to at least one of the first jaw or the second jaw.

In aspects, the surgical clip may further include an elongated collar configured to be detachably coupled to a surgical instrument. The first and second jaws may be coupled to the collar.

In aspects, the spherical magnet may define a channel therethrough, and the tether may extend through the channel.

In aspects, the tether may be a closed loop or a single strand.

In aspects, the tether may be directly attached to the first jaw.

In aspects, the tether may extend through a pair of openings defined in the first jaw. The pair of openings may define an axis therebetween that is parallel with a length of the first jaw.

In aspects, the tether may have two ends crimped to one another, such that the tether is a closed loop.

In aspects, the crimp may be disposed at a location between a point of attachment of the tether to the first jaw and a point of attachment of the tether to the spherical magnet.

In aspects, the spherical magnet may be fabricated from neodymium and coated with parylene.

In aspects, the suture may be fabricated from polypropylene.

In aspects, the tether may be directly attached to the spherical magnet.

In aspects, the tether may be a suture.

In aspects, each of the first and second jaws and the tether may be free from magnetically-attractive material.

In aspects, the tether may be free from magnetically-attractive material.

In accordance with another aspect of the disclosure, a surgical system for performing an endoscopic submucosal dissection is provided and includes a first surgical clip and a second surgical clip. Each of the first and second surgical clips includes first and second jaws configured to move between an open configuration and a closed configuration to grasp tissue therebetween. The first surgical clip includes a spherical magnet and a tether coupling the spherical magnet to the first jaw and/or second jaws. The second surgical clip includes a spherical body fabricated from a magnet or a metal and a tether coupling the spherical body to the first jaw and/or second jaw of the second surgical instrument.

### BRIEF DESCRIPTION OF THE DRAWINGS

The accompanying drawings, which are incorporated in and constitute a part of the specification, illustrate embodiments of the disclosure and, together with a general description of the disclosure given above as well as the detailed description of the embodiment or embodiments given below, serve to explain the principles of this disclosure.

FIG. 1 is a side, perspective view of a hand-held surgical instrument for deploying a surgical clip in an endoscopic submucosal dissection surgical procedure;

FIG. 2 is a side, perspective view of a distal end of the surgical instrument of FIG. 1 deploying a surgical clip through an endoscopic introducer;

FIG. 3 is a side, perspective view illustrating the surgical clip of FIG. 2 attached to the surgical instrument; and

FIG. 4 is a perspective view illustrating first and second surgical clips secured to one another by their respective magnets during an endoscopic submucosal dissection.

### DETAILED DESCRIPTION

The present disclosure is generally directed to a surgical system including first and second surgical clips used in performing an endoscopic submucosal dissection. The first and second surgical clips are each equipped with a magnet. During device use, the two (or more) magnets will be connected between the lesion side and anchor side at an unknown angle. The angle may depend on many factors, such as lesion size, target organ, and physician placement of the anchoring magnet. Magnet to magnet attachment strength is a critical design output for proper clinical performance. If magnets detach from each other at too low of a force, the tissue will not be able to be lifted to obtain traction and dissection plane visualization. If magnet to magnet attachment strength is too high, it can be higher than the attachment strength of the anchoring grasper to tissue. If a physician were to overextend the lumen, it is desired for the magnets to disengage before the graspers detach from the tissue to prevent injury to the tissue such as perforation, tissue laceration, etc. An additional advantage is that re-connecting the magnets is a simple clinical maneuver as compared to having to get a new device and reattach to the wall. Because of the two-sided performance limits, magnet to magnet attachment strength must be predictable in all clinical use.

Magnet to magnet detachment can occur with 3 distinct failure modes: tensile, shear, and peel. These three failure modes have very different force values and create a wide and inconsistent population for magnet to magnet strength. The highest forces are obtained with tensile, but this only occurs when the magnets are pulled with a straight vector along their longitudinal axes. Once the force vector is off axis (angled) the peel and shear failure modes occur randomly. Having this variability in magnet to magnet strength does not allow prior designs to meet the two-sided performance limits described above.

Accordingly, the present application provides a spherical magnet designed to overcome the above issues. The spherical magnet ensures contact at a specific point no matter the vector of tensile load and allows the magnet to rotate such that the force vector is always along the magnets' longitudinal axes. The failure mode is always a tensile failure and the magnet to magnet attachment strength is a very consistent population that easily meets the two sided performance limits. These and other aspects of the present disclosure are described in greater detail below.

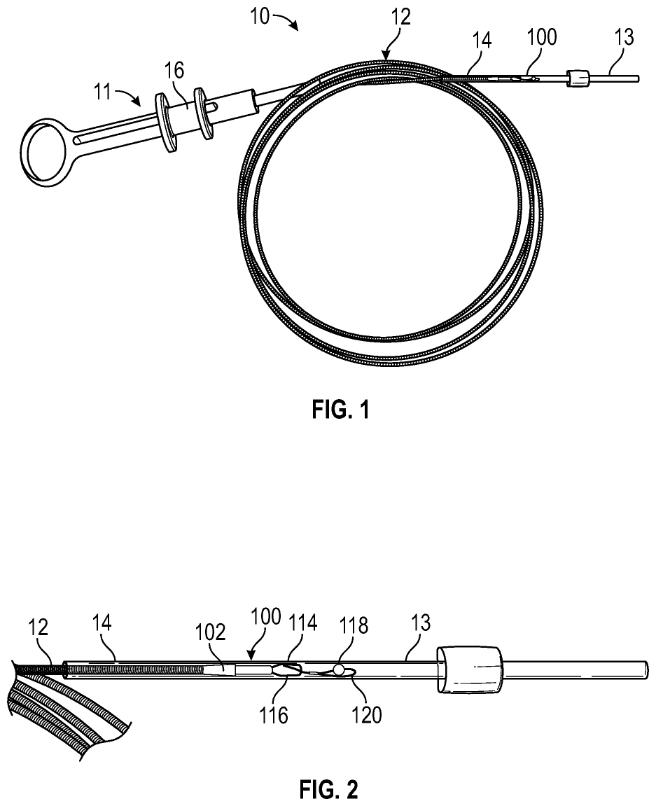

FIG. 1 illustrates a surgical system including a hand-held surgical instrument 10 for deploying a surgical clip 100 a second surgical clip 200 (FIG. 4). The surgical instrument 10 generally includes a handle 11, a flexible shaft 12 extending distally from the handle 11, and a surgical clip 100 detachably coupled to a distal end portion 14 of the shaft 12. In some aspects, instead of the clip 100 being deployable from a hand-held instrument, the clip 100 may be deployed from a surgical robotic arm.

The surgical instrument 10 includes an actuation mechanism, such as, for example, a puller (not explicitly shown) axially movable within the shaft 12. The puller may be a wire, cable, or the like. The puller may have a proximal end operably coupled to a trigger 16 of the handle 11 such that an actuation of the trigger 16 proximally translates the puller. The puller may have a distal end detachably coupled to the surgical clip 100 such that proximal translation of the puller moves the surgical clip 100 from an open configuration to a closed configuration and for ultimately releasing the surgical clip 100 from the remainder of the surgical instrument 10, as will be described. It is contemplated that the surgical instrument 10 may include any suitable actuation mechanism for deploying the surgical clip 100, such as a drive rod or a drive tube.

It is contemplated that the first and second surgical clips 100, 200 are the same or substantially similar. Therefore, only details with respect to the first surgical clip 100 will be provided herein.

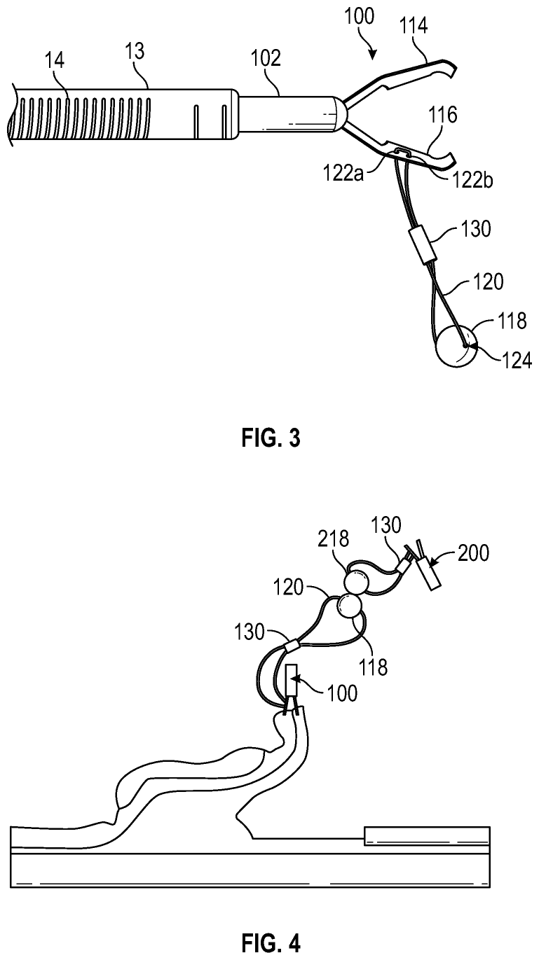

With reference to FIGS. 2 and 3, the surgical clip 100 has a proximal body portion 102, such, as for example, an elongated collar, a pair of opposing jaws 114, 116, a tether 120, and a magnet 118. The collar 102 is detachably coupled to the distal end portion 14 of the shaft 12, and the pair of jaws 114, 116 is movably received in the collar 102. In aspects, the shaft 12 may have a release latch (not shown) coupled to the collar 102 whereby the puller actuates the release latch to deploy the first surgical clip 100 from the shaft 12.

The pair of jaws 114, 116 is movable between an open state and a closed state. In the open state, the jaws 114, 116 are spaced from one another, and in the closed state the jaws 114, 116 are approximated toward one another. Each of the pair of jaws 114, 116 has a proximal end portion received in the collar 102, and a distal end portion configured to grasp tissue between the jaws 114, 116. The jaws 114, 116 are resiliently biased toward the open state. In some aspects, the jaws 114, 116 may be resiliently biased toward the open state by a biasing member (not shown) such as, for example, a spring. Alternately, the jaws 114, 116 may be resiliently biased toward the closed state. The jaws 114, 116 are axially movable relative to the collar 102 from a proximal position, in which the distal end portion of the jaws 114, 116 are approximated toward one another, and a distal position, in which the distal end portion of the jaws 114, 116 are spaced from one another. In aspects, the jaws 114, 116 may be detachably coupled to a distal end portion of the puller and axially movable relative to the collar 102 by the puller.

The tether 120 may be a suture for coupling the magnet 118 to the jaw 116. In aspects, the tether 120 may be a single strand tether, a flexible wire, thread, cable, a polymer or rubber strand, or the like. The tether 120 may be fabricated from polypropylene and/or coated with a lubricious material, such as silicone, to aid axial rotation of the magnet 118. The tether 120 may have a looped configuration and extends through a pair of openings 122a, 122b in the second jaw 116 and a channel 124 defined through the magnet 118. The openings 122a, 122b in the second jaw 116 may be arranged along the length of the second jaw 116 (e.g., the openings 122a, 122b define an axis therebetween that is parallel with the length of the second jaw 116) to inhibit rotation of the surgical clip 100 when in use. The tether 120 may have two ends attached to one another via a metal crimp 130, such that the tether 120 is a closed loop. The crimp 130 may be disposed at a location between a point of attachment of the tether 120 to the jaw 116 and a point of attachment of the tether 120 to the spherical magnet 118. In aspects, the crimp 130 may give the tether 120 an overall figure eight-shape. In aspects, the crimp 130 may be placed in a location along the length of the tether 120 closer to the magnet 118 than to the jaw 116, such that the distal loop of the figure eight-shape is smaller than its proximal loop.

The tether 120 extends through the channel 124, which is defined through a circumferential outer surface of the magnet 118. In some aspects, the tether 120 may extend through any suitable portion of the magnet 118, such as the radial center thereof such that the channel 124 is defined through a diameter of the magnet 118. The tether 120 may be slidable through the channel 124 of the magnet 118 to allow the magnet 118 to slide along the tether 120 and/or the magnet 118 may be rotatable about an axis defined by the channel 124. In aspects, the magnet 118 may be fixed to the tether 120.

The magnet 118 has a spherical shape and may be made of, coated with, or contain neodymium, iron, or any other suitable permanent magnetic element or electromagnet. The magnet 118 may be further coated with parylene, PTFE (or any suitable biocompatible lubricious material), plated with a material such as a precious metal (e.g., gold, platinum, etc.), or another suitable material to encase the magnet. The spherical shape of the magnet 118 ensures contact with another spherical body 218 (FIG. 4) of a second surgical clip 200. The spherical body 218 of the second surgical clip 200 may be a permanent magnet, like magnet 118, or a metal material that is magnetically attracted to magnet 118 (e.g., iron). Since the magnet 118 of the first surgical clip 100 and the spherical body 218 of the second surgical clip 200 are magnetically attracted to one another and are both spherical in shape, this ensures contact with one another at a specific point no matter the vector of tensile load and allows the magnet 118 and spherical body 218 to rotate such that the force vector is always along the magnet's 118 and spherical body's 218 longitudinal axes. The spherical shape of the magnet 118 and spherical body 218 and the extending of tethers 120 through the radial centers of the magnet 118 and spherical body 218 such that the channels 124 are defined through diameters of the magnet 118 and the spherical body 218 allow for the magnet 118 and the spherical body 218 to slide and roll along one another's outer surfaces during a surgical procedure without disengaging despite experiencing shear and/or peeling forces. Likewise, in some embodiments, the lengthwise arrangement of the openings 122a, 122b, the figure eight-shape of tether 120 and the location of the crimp 130 closer to magnet 118 and spherical body 218 than to jaws 116 allow for the magnet 118 and spherical body 218 to slide and roll along one another's outer surfaces during a surgical procedure without disengaging despite experiencing shear and/or peeling forces.

In aspects, the magnet 118 may having a surface feature, such as, for example, a projection, and the spherical body 218 may have a corresponding surface feature, such as, for example, a depression configured for receipt of the projection to enhance the engagement between the magnet 118 and the spherical body 218. In aspects, the outer surface of each of the magnet 118 and the spherical body 218 may be polished to facilitate the relative rolling/sliding motion between the magnet 118 and spherical body 218.

The jaws 114, 116, the tether 120, and/or the collar 102 may be fabricated from a non-magnetic material or low-magnetic material, such that the magnet 118 is free from being magnetically attracted to components of the surgical clip 100. For example, the jaws 114, 116, the tether 120, and/or the collar 102 may be fabricated from cobalt. In some aspects, the jaws 114, 116, the tether 120, and/or the collar 102 may be fabricated from a non-metallic material, such as, for example, plastics. In some aspects, the jaws 114, 116, the tether 120, and/or the collar 102 may be demagnetized via a processing (e.g., pickling or annealing).

In use, the first surgical clip 100 including the jaws 114, 116, the tether 120, and the magnet 118, are passed through a channel in an endoscope. In some aspects, an endoscopic introducer 13 (FIG. 1) may be provided that is passed through the endoscope channel and through which the surgical clip 100 is passed. Due to the spherical shape of the magnet 118, the magnet 118 easily slides through the endoscope and/or the introducer 13 without getting lodged in place.

Upon the jaws 114, 116 exiting the channel of the endoscope and/or introducer 13, the resilient bias of the jaws 114, 116 causes the jaws 114, 116 to move to the open state. With the first surgical clip 100 coupled to the shaft 12 and the jaw members 114, 116 in the open configuration, the first surgical clip 100 is positioned adjacent tissue (e.g., a lesion). The tissue is positioned between the jaw members 114, 116, whereupon the puller of the surgical instrument 10 retracts the jaw members 114, 116 proximally through the proximal body portion 102. An inner wall of the proximal body portion 102 acts on the jaw members 114, 116 to move the jaw members 114, 116 toward the closed configuration about the tissue. With the tissue grasped between the jaw members 114, 116, the puller is further retracted to detach the puller from the jaw members 114, 116, thereby releasing the proximal body portion 102 of the first surgical clip 100 from the shaft 12 and leaving the first surgical clip 100 at the surgical site. Other mechanisms for releasing the first surgical clip 100 from the shaft 12 are also contemplated.

The second surgical clip 200 may be deployed into the surgical site in a similar manner and coupled to selected tissue, such as, for example, a muscle layer of a gastric, colonic, or esophageal wall. With the two surgical clips 100, 200 attached to targeted tissue, as shown in FIG. 4, the magnets 118, 218 of the surgical clips 100, 200 attract one another, thereby providing counter-traction in the lesion to assist in dissection of the lesion. In some aspects, a third surgical clip (not shown), similar to the surgical clips 100, 200, may be provided to maintain traction mid-procedure. For example, during dissection of the lesion, to ensure that constant traction is being applied to the lesion, a third surgical clip may be deployed adjacent the first surgical clip 100 to switch the connection of the first surgical clip 100 from the second surgical clip 200 to the third surgical clip. The third surgical clip is positioned at a location selected to adjust the angle of traction applied to the lesion. It is contemplated that additional surgical clips may be deployed progressively to maintain traction throughout the process of dissection.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

What is claimed is:

1. A surgical system for performing an endoscopic submucosal dissection, comprising:
a first surgical clip including:
first and second jaws configured to move between an open configuration and a closed configuration to grasp tissue therebetween;
a spherical magnet; and
a tether coupling the spherical magnet to at least one of the first or second jaws;
a second surgical clip including:
first and second jaws configured to move between an open configuration and a closed configuration to grasp tissue therebetween;
a spherical body fabricated from a magnet or a metal; and
a tether coupling the spherical body to at least one of the first or second jaws of the second surgical clip.

2. The surgical system according to claim 1, wherein the spherical magnet of the first surgical clip defines a channel therethrough, the tether of the first surgical clip extending through the channel.

3. The surgical system according to claim 2, wherein the tether of the first surgical clip is a closed loop or a single strand.

4. The surgical system according to claim 1, wherein the tether of the first surgical clip extends through a pair of openings defined in the first jaw of the first surgical clip, the pair of openings defining an axis therebetween that is parallel with a length of the first jaw of the first surgical clip.

5. The surgical system according to claim 4, wherein the tether of the first surgical clip has two ends crimped to one another, such that the tether is a closed loop.

6. The surgical system according to claim 5, wherein the crimp is disposed at a location between a point of attachment of the tether of the first surgical clip to the first jaw of the first surgical clip and a point of attachment of the tether of the first surgical clip to the spherical magnet.

7. The surgical system according to claim 1, wherein the first surgical clip includes an elongated collar configured to be detachably coupled to a surgical instrument, wherein the first and second jaws of the first surgical clip are coupled to the collar.

8. The surgical system according to claim 1, wherein each of the spherical magnets is fabricated from neodymium and coated with parylene.

9. The surgical system according to claim 8, wherein each of the tethers is fabricated from polypropylene.

10. The surgical system according to claim 1, wherein the tether of the first surgical clip is directly attached to the spherical magnet of the first surgical clip.

11. The surgical system according to claim 1, wherein each of the tethers is a suture.

12. The surgical system according to claim 1, wherein each of the first and second jaws and the tether of the first surgical clip are free from magnetically-attractive material.

13. The surgical system according to claim 1, wherein the tether of the first surgical clip is free from magnetically-attractive material.

14. A surgical clip for use in endoscopic submucosal dissection, comprising:
first and second jaws configured to move between an open configuration and a closed configuration to grasp tissue therebetween;
a spherical magnet; and
a tether coupling the spherical magnet to at least one of the first jaw or the second jaw.

15. The surgical clip according to claim 14, further comprising an elongated collar configured to be detachably coupled to a surgical instrument, wherein the first and second jaws are coupled to the collar.

16. The surgical clip according to claim 14, wherein the spherical magnet defines a channel therethrough, the tether extending through the channel.

17. The surgical clip according to claim 16, wherein the tether is a closed loop or a single strand.

18. The surgical clip according to claim 14, wherein the tether is directly attached to the first jaw.

19. The surgical clip according to claim 18, wherein the first jaw defines a pair of openings therethrough, the tether being received in the pair of openings, the pair of openings defining an axis therebetween that is parallel with a length of the first jaw.

20. The surgical clip according to claim 19, wherein the tether has two ends crimped to one another, such that the tether is a closed loop.
